(19) United States
(12) Patent Application Publication
(10) Pub. No.: US 2015/0202167 A1
(43) Pub. Date: Jul. 23, 2015

(54) Trans-Clomiphene Formulations and Uses Thereof

(71) Applicant: REPROS THERAPEUTICS INC., The Woodlands, TX (US)

(72) Inventors: Joseph S. Podolski, The Woodlands, TX (US); Kuang Hsu, The Woodlands, TX (US)

(73) Assignee: REPROS THERAPEUTICS INC., The Woodlands, TX (US)

(21) Appl. No.: 14/421,119

(22) PCT Filed: Mar. 15, 2013
(86) PCT No.: PCT/US2013/032659

§ 371 Date: Feb. 11, 2015

Related U.S. Application Data
(60) Provisional application No. 61/691,722, filed on Aug. 21, 2012.

(51) Int. Cl. A61K 31/138; A61K 9/50; A61K 9/16

(52) U.S. Cl. CPC A61K 31/138; A61K 9/16; A61K 9/50

(57) ABSTRACT

The present invention provides iraws-clomiphene and pharmaceutically acceptable salts and solvates thereof, characterized in that trans-clomiphene is in particulate form having a specific size range. The invention is also directed to pharmaceutical compositions comprising or formulated using trans-clomiphene or pharmaceutically acceptable salts and solvates having a specified size range and their use in treating disorders including secondary hypogonadism, type 2 diabetes, elevated cholesterol, elevated triglycerides, wasting, lipodystrophy, female and male infertility, benign prostate hypertrophy, prostate cancer, breast cancer, ovarian cancer and endometrial cancer.

### CROSS-REFERENCE TO RELATED APPLICATIONS

This application claims the benefit of U.S. Provisional Application No. 61/691,722, filed Aug. 21, 2012, the contents of which are incorporated herein by reference.

### FIELD OF THE INVENTION

The present invention relates to trans-clomiphene, in particulate form, useful for treatment of various hormone-dependent disorders. More particularly, the trans-clomiphene is of a particle size range that provides an enhanced bioavailability.

### BACKGROUND

Clomiphene is a selective estrogen receptor modulator related to tamoxifen. Clomiphene binds to estrogen receptors and blocks the normal estrogen feedback on the hypothalamus and subsequent negative feedback on the pituitary. This leads to increases in luteinizing hormone (LH) and follicle stimulating hormone (FSH). In men, these increased levels of gonadotropins stimulate the Leydig cells of the testes and result in the production of higher testosterone levels. For example, Tenover et al., J. Clin. Endocrinol. Metab. 64:1103, (1987) and Tenover et al., J. Clin. Endocrinol. Metab. 64:1118 (1987) found increases in FSH, LH in both young and old men after treatment with clomiphene. They also found increases in free and total testosterone in men with young men showing significant increases.

In females, clomiphene is currently approved as a mixture of both cis- and trans-isomers, the cis-isomer being present as about 30% to 50% (Merck Manual) for the induction of ovulation in anovulatory women. The increases in LH and FSH in anovulatory females following administration of clomiphene result in follicular growth and ultimately ovulation. The drug is recommended to be administered for 5 days at a dose of up to 100 mg daily

Ernst et al., J. Pharmaceut. Sci. 65:148 (1976), have shown that clomiphene is a mixture of two geometric isomers which they refer to as cis, -Z-, clomiphene (cis-clomiphene or zuclomiphene) and trans-, E-, clomiphene, (trans-clomiphene or enclomiphene). According to Ernst, et al. trans-clomiphene HCl has a melting point of 149° C.-150.5° C., while cis-clomiphene HCl has a melting point of 156.5° C.-158° C. Ernst et al. have also noted that (the trans-isomer) is antiestrogenic (AE) while the cis-isomer is the more potent and more estrogenic form and has also been reported to have anti-estrogenic activity. The authors attribute the effect of the drug on ovulatory activity to both forms stating that the mixture is more effective than trans-clomiphene alone. The trans-isomer aids ovulation at the level of the hypothalamus. The estrogenic isomer cis-clomiphene contributes to enhanced ovulation elsewhere in the physiologic pathway leading to ovulation. The isomers are also reported to have different in vivo half-life. The cis isomer has been reported to leave residual blood levels for in excess of one month following a single dose.

Clomiphene has been associated with numerous side effects including: blurred vision, abdominal discomfort, gynecomastia, testicular tumors, vasomotor flushes, nausea, and headaches. Furthermore, other studies suggest that clomiphene possesses both genotoxic and tumor enhancement effects. The net outcome of these observations is that clomiphene in its current format, having between 30% and 50% of the cis isomer, would be unacceptable for chronic therapy in men for the treatment of testosterone deficiency.

Oral administration of trans-isomer of clomiphene (trans-clomiphene or enclomiphene) has been demonstrated to be effective in the treatment of a panoply of disorders ranging from secondary hypogonadism in males to induction of ovulation in anovulatory females. An improvement in the physical characteristics of trans-clomiphene would potentially offer a more beneficial therapy.

### SUMMARY

The present invention provides trans-clomiphene, characterized in that the trans-clomiphene is in particulate form, said particles having a mean particle size of less than about 30 microns, and preferably between about 5 and 20 microns.

Further, the present invention encompasses trans-clomiphene, wherein at least 90% of the particles have a particles size of less than about 50 microns.

Pharmaceutical compositions comprising or formulated using trans-clomiphene in particulate form are also provided. The compositions are used for treating a variety of disorders including, without limitation, secondary hypogonadism, type 2 diabetes, elevated cholesterol, elevated triglycerides, wasting, lipodystrophy, female and male infertility, benign prostate hypertrophy, prostate cancer, breast cancer, uterine cancer and ovarian cancer.

### BRIEF DESCRIPTION OF THE DRAWING

FIG. 1 shows the chemical structure of trans-clomiphene.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

It is to be understood that any ranges, ratios and ranges of ratios that can be formed by any of the numbers or data present herein represent further embodiments of the present invention. This includes ranges that can be formed that do or do not include a finite upper and/or lower boundary. Accordingly, the skilled person will appreciate that many such ratios, ranges and ranges of ratios can be unambiguously derived from the data and numbers presented herein and all represent embodiments of the invention.

Before the present compounds, compositions and methods are disclosed and described, it is to be understood that the terminology used herein is for the purpose of describing particular embodiments only and is not intended to be limiting. It must be noted that, as used in the present specification and the appended claims, the singular forms “a,” “an” and “the” include plural referents unless the context clearly dictates otherwise.

The term “oral” administration means that the active agent is in a formulation designed to be ingested, i.e. designed to be delivered to the gastrointestinal system for absorption.

The term “effective dosage” means an amount of the composition's active component sufficient to treat a particular condition.

The term “treat” or “treatment” as used herein refers to any treatment of any progesterone-dependent disorder or disease, and includes, but is not limited to, inhibiting the disorder or disease arresting the development of the disorder or disease; relieving the disorder or disease, for example, causing regression of the disorder or disease; or relieving the condition caused by the disease or disorder, relieving the symptoms of the disease or disorder.

The term “prevent” or “prevention,” in relation to a progesterone-dependent disorder or disease, means preventing the onset of disorder or disease development if none had occurred, or preventing further disorder or disease development if the disorder or disease was already present.

The term “pharmaceutically acceptable salt” refers to a salt prepared from a pharmaceutically acceptable non-toxic inorganic or organic acid. Inorganic acids include, but are not limited to, hydrochloric, hydrobromic, hydroiodic, nitric, sulfuric, and phosphoric. Organic acids include, but are not limited to, aliphatic, aromatic, carboxylic, and sulfonic organic acids including, but not limited to, formic, acetic, propionic, succinic, benzoic camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, alginic, and galacturonic acid. A preferred salt is the citrate salt.

The term “solvate” represents an aggregate that comprises one or more molecules of the solute, trans-clomiphene, with a molecule of solvent.

The term “mean particle size” is defined as equivalent spherical diameter as determined by laser light diffraction scattering.

In various embodiments, the present invention provides trans-clomiphene with a particle size within the specified narrow range. Control of the particle size within the specified narrow range provides a beneficial in vivo bioavailability.

The mean particle size of trans-clomiphene according to the present invention is less than about 30 microns, preferably between about 5 and about 20 microns. Further, the invention encompasses trans-clomiphene with at least 90% of the particles having a particle size of less than about 50 microns. More preferably, the mean particle size range is between about 5 and about 20 microns with at least 90% of the particles having a size of less than about 35 microns.

The invention also provides pharmaceutical compositions comprising or formulated using trans-clomiphene with a particle size within the specified narrow range and one or more pharmaceutically acceptable carriers.

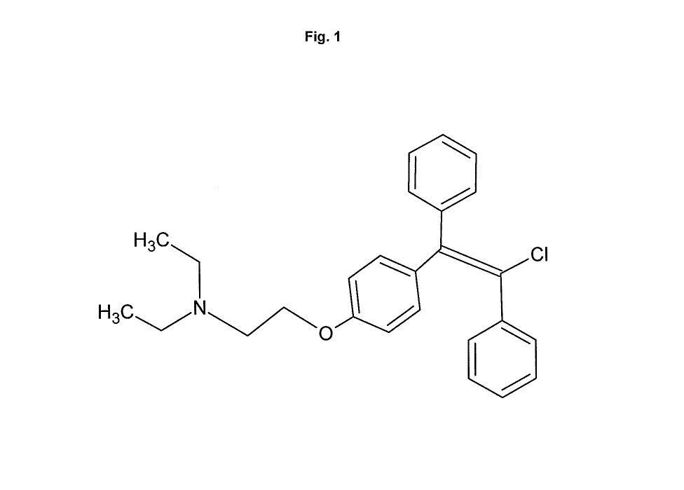

Trans-clomiphene's chemical name is trans-2-(p-(2-chloro-1,2-diphenylvinyl)phenoxy)triethylamine (or 2-[4-(2-chloro-1,2-diphenylethenyl)phenoxy]-N,N-diethylethanamine) The chemical structure is illustrated at FIG. 1. “Trans-clomiphene” also encompasses the salts and solvates thereof, with the citrate salt being preferred. Trans-clomiphene is a selective estrogen receptor modulator (SERM) which is believed to interfere at a hypothalamic level with steroid feedback inhibition of gonadotropin secretion thereby increasing the release of FSH and LH.

Trans-clomiphene can be made according to established procedures. U.S. Pat. No. 2,914,563 describes the preparation of clomiphene and is incorporated herein by reference in its entirety. U.S. Pat. No. 3,848,030 describes a method to separate the cis- and trans-isomers of clomiphene, and is incorporated herein by reference in its entirety.

Trans-clomiphene within a mean particle size of less than about 30 microns, preferably between about 5 and about 20 microns, can be administered by a variety of routes including but not limited to, intravenous, subcutaneous, buccal, transmucosal, intrathecal, intradermal, intracisternal, intramuscular, transdermal, intraperitoneal, epidural, vaginal, rectal, intranasal, sublingual, intra-articular, intra-cerebrospinal and intrasynovial, although, oral administration is the preferred route. Thus, another aspect of the present invention is a pharmaceutical composition comprising an effective amount of trans-clomiphene or a pharmaceutically acceptable salt thereof, and a pharmaceutically acceptable carrier, diluent, or excipient.

Trans-clomiphene may be present in the pharmaceutical composition between 0.1% and 99.9% by weight of the formulation and may be the only active agent in the composition or may be combined with one or more additional active agents, depending on the intended use of the composition. The composition may comprise trans-clomiphene at a dosage between about one mg to about 200 mg (although the determination of optimal dosages is with the level of ordinary skill in the art). The composition may comprise trans-clomiphene at a dosage of about 1 mg, 2 mg, 3, mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg or there between. Preferably, the composition comprises trans-clomiphene at a dosage of between 5 and 100 mg, e.g. at a dosage of 12.5 mg, 25 mg or 50 mg. By “pharmaceutically acceptable” it is meant the carrier, diluent, excipient and salt must be compatible with the other ingredients of the formulation and not deleterious to the recipient thereof.

Pharmaceutical compositions of the present invention can be prepared by any procedure known in the art using readily available and well known ingredients. For example, trans-clomiphene can be formulated with common excipients, diluents or carriers and formed into tablets, capsules, suspensions, powders and the like. Examples of excipients, diluents and carriers suitable for such formulations include, without limitation, filler and extenders such as strach, sugars, mannitol and silicic derivatives; binding agents such as carboxymethyl cellulose and other cellulose derivatives, alginates, gelatin and polyvinylpyrrolidone; moisturizing agents such as glycerol; disintegrating agents such as calcium carbonate, sodium bicarbonate and cross-linked povidone; agents for retarding dissolution such as paraffin; resorption accelerators such as quaternary ammonium compounds; surface active agents such as cetyl alcohol, polysorbate 80, glyercol monstearate; adsorptive carriers such as kaolin and bentonite; and lubricants such as talc, calcium and magnesium stearate and solid polyethyl glycols.

The pharmaceutical compositions are useful to increase testosterone e.g. in secondary hypogonadal males to treat the secondary hypogonadism or to treat a disorder related thereto such as, without limitation, oligospermia, azoospermia, wasting and depression as described in U.S. Pat. No. 7,759,360, the entire content of which is hereby incorporated by reference. The pharmaceutical compositions are also useful to decrease cholesterol levels as described in U.S. Pat. No. 7,368,480, the entire contents of which are hereby incorporated by reference. The pharmaceutical compositions can also be used to prevent or treat a condition selected from the group consisting of benign prostate hypertrophy, prostate cancer and elevated triglycerides as described in US Patent Application Publication No. 2008/0242726, the entire contents of which are hereby incorporated by reference. The pharmaceutical compositions can also be used to prevent the transition from metabolic syndrome to type 2 diabetes or to treat type 2 diabetes or to reduce fasting glucose levels as described in US Patent Application Publication No. 2009/0099265, the entire contents of which are hereby incorporated by reference. The pharmaceutical compositions can also be used to treat female infertility in which case the composition is preferably administered to an anovulatory female as a daily dose in the early follicular phase of the menstrual cycle for five consecutive days. The pharmaceutical compositions are also useful for treating and/or preventing breast cancer and/or as an adjuvant therapy following initial treatment with surgery in order to minimize the possibility of relapse. The pharmaceutical compositions are also useful for treating endometrial (or uterine) cancer and ovarian cancer.

All of the references discussed herein are incorporated by reference in their entirety.

The following Examples are meant to be illustrative of the invention and are not intended to limit the scope of the invention as set out is the appended claims.

### Example 1

### Preparation of Trans-Clomiphene Citrate

Clomiphene citrate was prepared as follows:

A mixture of 20 g of 1-[p-(β-diethylaminoethoxy)phenyl]-1,2-diphenylethanol in 200 cc of ethanol containing an excess of hydrogen chloride was refluxed 3 hours. The solvent and excess hydrogen chloride were removed under vacuum, and the residue was dissolved in a mixture of ethyl acetate and methylene chloride. 1-[p-(β-diethylaminoethoxy)phenyl]-1,2-diphenylethylene hydrochloride was obtained, melting at 148 to 157 C. This hydrochloride salt was treated with N-chlorosuccinimide in dry chloroform under reflux. The product then obtained was converted to the free base and treated with citric acid. The di-hydrogen citrate salt of 1-[p-(β-diethylaminoethoxy)phenyl]-1,2-diphenylchloroethylene was obtained, melting at 116.5 to 118 C. Clomiphene citrate obtained by this process comprises between 30 and 50% cis-isomer and between 70 and 50% trans-isomer.

Trans-clomiphene was then separated from the racemic mixture of clomiphene isomers using the process described in Examples 31 and 32 of U.S. Pat. No. 3,848,030.

### Example 2

### Particle Size Analysis

Trans-clomiphene is characterized for size using an instrument adapted to measure equivalent spherical volume diameter, such as a Malvern Mastersizer 2000 laser diffraction particle size analyzer or equivalent instrument. After being characterized for size, the trans-clomiphene is then milled, if necessary, preferably using a pin mill under suitable conditions of mill rotation rate and feed rate, to bring the particle size value within the above mentioned limits according to the invention. The efficiency of milling is checked by sampling using a Malvern Mastersizer 2000 laser diffraction particle size analyzer and the final particle size is checked in a similar manner.

Trans-clomiphene in its particulate form within the above mentioned limits according to the invention may then be mixed with an excipient or carrier as necessary and for example used to fill capsules. Because the particles before or after milling are irregular in shape, it is necessary to characterize them by measurement of a property of the particles related to the sample property possessed by a theoretical spherical particle. The particles are thus allocated an “equivalent spherical diameter.”

The values found from characterizing a large number of “unknown” particles can be plotted frequency vs. diameter. This gives a characteristic curve representing size distribution of the sample, i.e., cumulative percentage under size distribution curve. Values from this can be read off directly or plotted on log-probability paper to give an appropriate straight line. The mean equivalent spherical volume diameter is the 50% undersize value. The mean equivalent spherical volume diameter found is thus a statistical representation of a theoretical particle having the same property as the “unknown” particle. The following is a description by way of example.

The particle size of trans-clomiphene citrate from Example 1 was analyzed. The refractive index of trans-clomiphene citrate was measured microscopically suing the Becke line method as described in McCrone W. C. et al., Polarized Light Microscopy, McCrone Research Institute, Chicago, 1984, pages 126-127. The sample refractive index was estimated to be 1.62 using this method.

0.1% (w/v) Lecithin in Isopar G™, 0.1% (w/v) Span™ 85 (sorbitane trioleate) in hexane, and 0.01% (w/v) Tween® 20 (polysorbate 20) in water were evaluated as dispersants for trans-clomiphene citrate. Samples suspended easily in each of the two organic dispersants and settled out of suspension slowly. Partial dissolution was observed in the sample suspended in aqueous dispersant, so 0.01% (w/v) Tween® 20 in water was unsuitable. Samples dispersed in the Isopar G™-based and hexane-based dispersants were examined microscopically. Sample composition and morphology were similar, revealing crystalline blades and needles between 10 and 150 μm in length, and differences in agglomeration were minor, with some soft agglomerates greater than 400 μm observed. 0.1% (w/v) Span™ 85 in hexane was selected for further analysis.

The sample absorption, or imaginary component of the refractive index, is a measure of the amount of light absorbed by the sample and is an important parameter in calculating a particle size distribution from a measured scattering pattern. The sample absorption cannot be measured experimentally so it must be estimated using “trial and error” using a scattering pattern for a particular compound. An initial particle size measurement was collected using the following parameters and particle size distributions were calculated from the scattering data using various sample absorption values:

| | |
|---|---|
| Refractive Index | 1.62 |
| Default Particle Absorption | 0.01 |
| Sample Measurement Time | 10 seconds |
| Background Measurement Time | 20 seconds |
| Default Pump Speed | 1000 rpm |
| Recirculation Time | 60 seconds |
| Model | General Purpose |
| Sensitivity | Normal |
| Particle Shape | Irregular |

The weighted residual is a measure of the goodness-of-fit between the measured data and a mathematical model that allows conversion of that data into a particle size distribution. A sample absorption index of 0.01 produced the best fit and was chosen for all subsequent particle size analyses.

Repeated particle size measurements of the sample dispersed in 0.1% (w/v) Span™ 85 in hexane were collected while recirculating over the course of approximately five minutes with a pump speed of 1000 rpm. Particle size (d10, d50, d90) was plotted versus the recirculation time. Values for both the d10 and d50 fell within a narrow range for the duration. The decrease in the d90 primarily occurred over the first 90 seconds with a gradual decrease thereafter. This decrease in the d90 suggested that agglomerates were dispersed within 90 seconds and some attrition may have occurred with longer recirculation so a recirculation time of 90 seconds was selected for further analyses.

Repeated particle size measurements of the sample were collected while recirculating for 90 seconds with increasing pump speeds to examine the effect of pump speed on particle size. Differences in the d10 and d50 were small, but the d90 showed an increase in particle size with a pump speed of 1500 rpm but no further increase at 2000 rpm. A pump speed of 1500 rpm was selected to optimize both dispersion of agglomerates and suspension of the larger particles and to minimize attrition of the blades and needles.

The suspended sample was recovered from the dispersion cell following particle size measurement with a pump speed of 1500 rpm and was microscopically examined Primary particles were reasonably well dispersed and photomicrographs were consistent with those collected prior to recirculation with similar numbers and sizes of blades and needles suggesting that attrition had been minimized.

The repeatability of the method was evaluated by making five replicate measurements using the final method conditions. The relative standard deviations for the d10, d50 and d90 were 2.60%, 3.42% and 10.70% respectively. All fell within the USP recommendation of ≦30%, ≦10% and ≦15% for the d10, d50 and d90 respectively.

One particle size measurement of each sample of trans-clomiphene citrate was collected using the final method conditions. Three of the lots shared a similar bimodal particle size distribution whereas one lot contained a third mode consisting of much larger particles. The remaining lot of was bimodal but reflected much larger particle sizes than the distributions of the earlier three lots with bimodal distributions.

Photomicrographs of each lot of trans-clomiphene citrate dispersed in 0.1% (w/v) Span™ 85 in hexane were collected following particle size measurement using final method conditions. The results were consistent with the particle size results.

The three lots sharing a similar particle size distribution also shared similar morphology primarily consisting of blades and needles 10-150 μm in length and a few equant particles ≦10 μm. Final conditions for determining particle size were:

| | |
|---|---|
| Sample Refractive Index | 1.62 |
| Sample Absorption | 0.01 |
| Dispersant | 0.1% (w/v) Span 85 in hexane |
| Dispersant Refractive Index | 1.39 |
| Sample Measurement Time | 10 seconds |
| Background Measurement Time | 20 seconds |
| Pump Speed | 1500 rpm |
| Recirculation Time | 90 seconds |
| Model | General Purpose |
| Sensitivity | Normal |
| Particle Shape | Irregular |

Refractive index determination was performed using a Leica DM LP microscope. A single, substage polarizer was used to view samples. Samples were placed on a glass slide, a coverslip was placed over the sample, and a drop of certified Cargill refractive index oil was added. The movement of the Becke line was observed while defocusing the sample.

Polarized light microscopy was performed using a Leica DM LP microscope equipped with a Spot Insight color camera. Crossed-polarized light was used with a first order red compensator. A 10×, 20× or 40× objective was used to view the sample. Images were acquired at ambient temperature using Spot Advanced software (v.4.5.9).

Particle size data was acquired using a Malvern Instruments MS2000 equipped with a Hydro 2000 μP dispersion unit. Data was collected and analyzed using Mastersizer 2000 v. 5.60 software using volume based measurements. NIST-traceable glass beads were used as the reference standard.

Particle size of trans-clomiphene citrate using the final method conditions is reproduced below:

| Lot No. | d10 (μm)^{a} | d50 (μm)^{b} | d90 (μm)^{c} |
|---|---|---|---|
| 31249 | 4.850 | 13.455 | 76.891 |
| 16204 | 8.058 | 106.743 | 318.464 |
| 24712 | 4.038 | 13.902 | 218.573 |
| 32305 | 3.373 | 9.664 | 44.995 |
| 24867 | 4.794 | 13.418 | 70.289 |

^{a}10% of the total volume of particles is less than the indicated particle size
^{b}50% of the total volume of particles is less than the indicated particle size
^{c}90% of the total volume of particles is less than the indicated particle size

The particle size distribution of trans-clomiphene from Lot No. 32305 is reproduced below:

| Size (μm) | Vol under % |
|---|---|
| 0.010 | 0.00 |
| 0.011 | 0.00 |
| 0.013 | 0.00 |
| 0.015 | 0.00 |
| 0.017 | 0.00 |
| 0.020 | 0.00 |
| 0.023 | 0.00 |
| 0.026 | 0.00 |
| 0.030 | 0.00 |
| 0.035 | 0.00 |
| 0.040 | 0.00 |
| 0.046 | 0.00 |
| 0.052 | 0.00 |
| 0.060 | 0.00 |
| 0.069 | 0.00 |
| 0.079 | 0.00 |
| 0.091 | 0.00 |
| 0.105 | 0.00 |
| 0.120 | 0.00 |
| 0.138 | 0.00 |
| 0.158 | 0.00 |
| 0.182 | 0.00 |
| 0.209 | 0.00 |
| 0.240 | 0.00 |
| 0.275 | 0.00 |
| 0.316 | 0.00 |
| 0.363 | 0.00 |
| 0.417 | 0.00 |
| 0.479 | 0.00 |
| 0.550 | 0.00 |
| 0.631 | 0.00 |
| 0.724 | 0.00 |
| 0.832 | 0.00 |
| 0.965 | 0.02 |
| 1.096 | 0.10 |
| 1.259 | 0.28 |
| 1.445 | 0.60 |
| 1.660 | 1.12 |
| 1.905 | 1.93 |
| 2.188 | 3.10 |
| 2.512 | 4.72 |
| 2.884 | 6.86 |
| 3.311 | 9.59 |
| 3.802 | 12.94 |
| 4.385 | 16.94 |
| 5.012 | 21.58 |
| 5.754 | 26.82 |
| 6.607 | 32.58 |
| 7.586 | 38.74 |
| 8.710 | 45.14 |
| 10.00 | 51.59 |
| 11.482 | 57.87 |
| 13.183 | 63.80 |
| 15.136 | 69.20 |
| 17.378 | 73.95 |
| 19.953 | 77.98 |
| 22.909 | 81.29 |
| 26.303 | 83.92 |
| 30.200 | 85.99 |
| 34.674 | 87.62 |
| 39.811 | 88.95 |
| 45.709 | 90.13 |
| 52.481 | 91.28 |
| 60.256 | 92.50 |
| 69.183 | 93.84 |
| 79.433 | 95.28 |
| 91.201 | 96.72 |
| 104.713 | 98.02 |
| 120.226 | 99.04 |
| 138.038 | 99.72 |
| 158.489 | 99.97 |
| 181.970 | 100.00 |
| 208.930 | 100.00 |
| 239.883 | 100.00 |
| 275.423 | 100.00 |
| 316.228 | 100.00 |
| 363.078 | 100.00 |
| 416.869 | 100.00 |
| 478.630 | 100.00 |
| 549.541 | 100.00 |
| 630.957 | 100.00 |
| 724.438 | 100.00 |
| 831.764 | 100.00 |
| 954.993 | 100.00 |
| 1096.478 | 100.00 |

Trans-clomiphene with the particle size distribution of the invention is expected to provide a consistent and improved in vivo absorption/bioavailability profile compared with trans-clomiphene having a particle size distribution outside the specified range. In addition to ensuring consistent delivery of trans-clomiphene to, and absorption from, the gastrointestinal tract, the specified particle size distribution provides better control during the manufacturing process. Controlling the particle size also minimizes variations in the quantity of water required to bring about the desired granulation.

Trans-clomiphene with the particle size distribution of the invention, alone or in combination with another active agent, generally will be administered in a convenient formulation. The following formulations are only illustrative and not intended to limit the scope of the invention.

### Example 3

### Formulations

Gelatin capsules comprising trans-clomiphene are prepared using the following:

| Component | Quantity (mg/capsule) |
|---|---|
| Trans-clomiphene citrate | 5.0-100 |
| Microcrystalline cellulose | 0-343.2 |
| Magnesium Stearate | 0-8- |

Trans-clomiphene, in crystal form, is blended with ⅓ of the total microcrystalline cellulose and passed through a mesh screen to ensure good distribution of the materials. The remaining ⅔ of the microcrystalline cellulose is then passed through a mesh screen and blended with the powder mixture. The resulting mixture is then milled through a suitable milling machine (e.g. a Comil® mill). Magnesium stearate, previously passed through a mesh screen, is added and mixed with the resulting granules. Following a uniformity analysis, the resulting mixture is encapsulated into gelatin capsules. A preferred gelatin capsule (size 3) formulation follows:

| Component | Quantity (mg/capsule) |
|---|---|
| Trans-clomiphene citrate | 12.5 |
| Microcrystalline cellulose | 85.5 |
| Magnesium Stearate | 2.0 |
| TOTAL | 100.0 |

Combination capsules comprising trans-clomiphene and an additional active agent (e.g. an aromatase inhibitor or oral testosterone) may be prepared according to the methods above:

| Component | Quantity (mg/capsule) |
|---|---|
| Trans-clomiphene citrate | 12.5 |
| Anastrole | 1.0-50.0 |
| Microcrystalline cellulose | 85.5 |
| Magnesium Stearate | 2.0 |
| TOTAL | 100.0 |
| Trans-clomiphene citrate | 12.5 |
| Testosterone Undecanoate | 80-200 mg |
| Microcrystalline cellulose | 85.5 |
| Magnesium Stearate | 2.0 |

Alternatively, tablets each containing 5.0 to 100 mg trans-clomiphene can be made as follows:

| Component | Quantity (mg/tablet) |
|---|---|
| Trans-clomiphene citrate | 5.0-100 |
| Starch | 30-60 |
| Polyvinylpyyrolidone | 0-8 |
| Microcrystalline cellulose | 25-45 |
| Magnesium Stearate | 0.1-2.0 |
| Talc | 0-3 |

For tablet formulations, trans-clomiphene, starch and cellulose are passed through a mesh sieve (e.g. No. 45) and mixed thoroughly. The solution of polyvinylpyrrolidone is mixed with the resultant powder which is then passed through a mesh sieve (e.g. No. 14). The granules produced are dried at 50-60 C and passed through a mesh sieve (e.g. No. 18). The magnesium stearate and talc, previously passed through a sieve (e.g. NO. 6) are then added to the granules which, after mixing, are compressed on a tablet machine to yield tablets.

Alternatively, suspensions each containing between 1.0 and 100 mg of trans-clomiphene per 5 ml dose are made as follows:

| Component | Quantity (mg/5 ml) | |
|---|---|---|
| Trans-clomiphene citrate | 1.0-100 | mg |
| Sodium carboxymethyl cellulose | 50 | mg |
| Syrup | 1.25 | mg |
| Benzoic acid solution | 0.10 | ml |
| Flavor | q.v. | |
| Color | q.v. | |
| Purified water to | 5 | ml |

For suspensions, trans-clomiphene is passed through a mesh sieve (e.g. No. 45) and mixed with the sodium carboxymethyl cellulose and syrup to form smooth paste. The benzoic acid solution, flavor, and color are diluted with some of the water and added, with stirring. Sufficient water is then added to produce the required volume.

Alternatively, suppositories may be prepared as follows:

| Component | Quantity (mg/suppository) |
|---|---|
| Trans-clomiphene citrate | 100-500 |
| Saturated fatty acid glycerides | 1000-3000 |

For suppositories, trans-clomiphene is passed through a mesh sieve (e.g. No. 60) and suspended in the saturated fatty acid glycerides previously melted using minimal heat necessary. The mixture is then poured into a suppository mold and allowed to cool.

1. The compound trans-clomiphene and pharmaceutically acceptable salts and solvates thereof, characterized in that the compound is in particulate form, said particles having a mean particle size of less than about 30 microns, at least about 90% of said particles have a size of less than about 50 microns.

2. The compound of claim 1 wherein said particles have a mean particle size of between about 5 and about 20 microns.

3. A compound of claim 1 which is a non-solvated crystalline form.

4. The compound of claim 1 wherein the compound is trans-clomiphene citrate.

5. A pharmaceutical formulation comprising or formulated using the compound of claim 1 and one or more pharmaceutically acceptable carriers, diluents or excipients.

6. The pharmaceutical formulation according to claim 5 wherein the compound is trans-clomiphene citrate.

7. The pharmaceutical formulation according to claim 5 wherein the formulation is a capsule.

8. The pharmaceutical formulation according to claim 7 comprising about 5 to about 100 mg trans-clomiphene citrate.

9. The pharmaceutical formulation according to claim 8 comprising 12.5 mg, 25 mg or 50 mg trans-clomiphene citrate.

10. The pharmaceutical formulation according to claim 7, further comprising microcrystalline cellulose and/or magnesium stearate.

11. The pharmaceutical formulation according to claim 10, each capsule comprising about 12.5 mg trans-clomiphene citrate, about 85.5 mg microcrystalline cellulose and about 2.0 mg magnesium stearate.

12. A method of treating secondary hypogonadism or a disorder associated therewith in a human male or treating and/or preventing type 2 diabetes in a human male or treating infertility in a human female or treating breast cancer, endometrial cancer, uterine cancer or ovarian cancer in a human female by administering to the male or female a pharmaceutical formulation according to claim 11.

13. The method according to claim 12, wherein the disorder associated with secondary hypogonadism is selected from the group consisting of reduction of muscle mass, reduction of bone density, reduction of libido, oligospermia, and azoospermia.

14. The method of claim 12, wherein the method comprises treating infertility in a human female.

15. The method according to claim 14, wherein the formulation is administered to an anovulatory female as a daily dose for a period of five consecutive days.

16. The method according to claim 12, wherein the method comprises treating and/or preventing type 2 diabetes in a human male.

17. The method according to claim 12, wherein the method comprises treating or preventing breast cancer in a human female.

18. The method according to claim 12, wherein the method comprises treating endometrial, uterine or ovarian cancer in a human female.
